(19) United States
(12) Patent Application Publication
(10) Pub. No.: US 2014/0335548 A1
(43) Pub. Date: Nov. 13, 2014

(54) SPERMIDINE BIOMARKER FOR THE DETECTION OF SULFOXIDIZING ACTIVITY

(71) Applicant: BIOSIGMA S.A., Santiago (CL)

(72) Inventors: Pilar Parada Valdecantos, Santiago (CL); Patricio Martinez Bellange, Santiago (CL)

(21) Appl. No.: 14/362,254

(22) PCT Filed: Nov. 29, 2012
(86) PCT No.: PCT/IB2012/056830

§ 371 Date: Jun. 2, 2014

(30) Foreign Application Priority Data
Dec. 2, 2011 (CL) 3066-2011

(51) Int. Cl. C12Q 1/04

(52) U.S. Cl. CPC C12Q 1/04
USPC 435/7.92; 435/34

(57) ABSTRACT

The invention refers to a method to evaluate the sulfoxidizing activity in a microbiologic process detecting the presence of the spermidine biomarker produced by the micro organisms that participate in such process.

### FIELD OF THE INVENTION

The invention refers to a method to evaluate the sulfoxiding activity in a microbiologic process detecting the presence of the biomarker spermidine produced by the microorganisms that participate in such process.

### BACKGROUND INFORMATION OF THE INVENTION

Within the microbiologic processes, such as bioleaching, the sulfoxidizing activity is fundamental whereby the components of the reduced sulphur present in the mineral, such as sulphides, tionates or elemental sulphur, among others, are oxidized to finally produce sulphuric acid. For one part, this process permits solubilizing directly the metals associated to sulphides in the mineral and on the other, by producing sulphuric acid it permits the conventional acid leaching of the oxidized mineral solubilizing the metals present in the mineral.

Within the sulfoxidizing microorganisms, the most common and widely found in industrial processes that involve the presence of sulphur or reduced compositions of sulphur is the Acidithiobacillus thioxidans.

The petitioner, Biosigma SA., has patented a particular strain of Acidithiobacillus thioxdans, the Licanantay DSM 1318 strain (see patent of Biosigma SA. CL 2001-05 or its equivalent U.S. Pat. No. 700,343 B2) which shows improved properties in the oxidization of sulphur components preventing the passivising of the minerals. The Licanantay DSN 173218 strain can be conveniently used in any microbiologic process, especially in any productive microbiologic process that requires sulfoxidizing activity.

Up to this moment the way to evaluate the sulfoxidizing activity in a microbiological process is by determining the quantity of sulfoxidizing micro organisms such as Acidithiobacillus thiooxidans, present in such process, for example in a reactor or in a bioleaching heap. It is complicated to evaluate the concentration of micro organisms, since it requires in the first place to isolate the present micro organisms and then the specifically identification of them. There are difficulties in all these stages as often these microorganisms are associated to solid mineral, to very acid solutions and with high concentrations of salts, which interfere with the standard microbiology or molecular biology processes, which must be adapted to be able to evaluate these micro organisms in particular. Additionally the process of specific identification normally is slow, so that it is no useful as a control parameter of immediate operation

The need then arises to have a method that will permit a rapidly evaluation of the sulfoxidizing activity in a microbiologic process.

The method of the invention solves this technical problem, providing a method that evaluates the sulfoxidizing activity determining the presence of the biomarker spermidine rapidly and specifically.

The inventors have found that surprisingly the sulfoxidizing activity can be correlated with the presence of spermidine in the supernatant of a culture or in the effluent of a microbiologic procedure.

The spermidine (NH₂—(CH₂)₄—NH—(CH₂)₃NH₂), is a polyamine that together with putrescine, are the most abundant and are widely distributed in all live beings. Their function has not been clearly determined but it is estimated that they are related with the protection of the nucleic acids and which are fundamental in the division, growth, differentiation and cellular death. In the thermophile microorganisms, such as some sulfoxidizing microorganisms, it is believed that spermidine participates in the protection of the nucleic acids and the proteins against the changes of temperature.

The inventors have determined that the sulfoxidizing bacteria such as Acidithiobacillus thiooxidans produce mostly spermidine while other gram negative bacteria produce putrescine.

Surprisingly the inventors have found that spermidine, that normally is found in intracellular form, is secreted by the sulfoxidizing micro organisms in important quantities to the supernatant or extracellular medium in the presence of insoluble sulphur compounds, and also, but to a lesser extent, in the presence of soluble sulphur compounds , such as for example tetrathionate. Without restricting ourselves by the theory it is possible that the spermidine secreted in the middle is a signal of quorum sensing for the formation of the necessary biofilm for the sulfoxidizing activity on the insoluble sulphur compositions. In accordance with the above, the method of the invention has been developed, evidencing the sulfoxidizing activity in a microbiologic process when is detected the presence of the spermidine biomarker

We have not found in the prior art the use of any document that describes a biomarker to determine the sulfoxidizing activity in a microbiologic process.

Likewise, spermidine has been used as biomarker but in applications that are very different from those of this invention. For example, Higuchi C M and Wang W. (J. Cell. Biochem. 1995 February; 57(2):256-61) describe the use of spermidine as biomarker of the abnormal proliferation of the colorectal mucosa as predictor of colorectal cancer Einspahr J G et. al. (Cancer Epidemiol Biomarkers Prev. 2006 October; 15(10):1841-8) determines the concentration of spermidine in biopsies of patients with skin sun damage and actinic keratosis as biomarker for the chemical prevention of skin cancer. Reynoso Orozco R., et al (Interciencia 2008 Ma 33(5) 384-388) describes the use of polyamines as biomarkers of tumoral activity and uses them to determine the antitutoral activity of Bursera fagaroides. In another scope Shoeb F. et al (Plant Science 2011 May; 160(6) 1229-1235) reveal the use of the proportion between putrescine and spermidine as biomarkers of the capacity of regeneration of the plants, specifically in rice plants. In general terms spermidine has been used as a biomarker of a proliferation activity and in environments very far from the microbiologic processes and of the sulfoxidizing activity.

### DESCRIPTION OF THE FIGURES

FIG. 1.

Mass spectrum from a solution with 5 ng/μL of spermidine; the characteristic peak is observed at 146, 1648 m/z.

FIG. 2.

Graph of spermidine detection in a reactor for production of biomass of Acidithiobacillus thiooxidans, strain Licanantay DSM 17318, with S elemental and in different times of cellular growth: exponential growth phase (T1), early stationary phase (T2) and late stationary phase (T3), as described in example 1.1.

FIG. 3.

Mass spectrum of the effluent from the column for sulphuric acid production described in example 2.1, where the characteristic peak of spermidine is observed at 146, 148 m/z.

### DESCRIPTION OF THE INVENTION

The invention refers to a method to evidence the sulfoxidizing activity in a microbiologic process detecting the presence of the biomarker spermidine in the extracellular medium. Spermidine, (NH₂—(CH₂)₄—NH—(CH₂)₃NH₂), is a molecule of biologic origin that can only be present in a process of oxidization of sulphur when being produced and secreted in the medium by the present sulfoxidizing micro organisms.

The inventors have found that the most important microorganism in sulfoxidizing processes, Acidithiobacillus thiooxidans, secretes spermidine in the extracellular medium in great quantities in the presence of insoluble compounds of sulphur such as sulphides, tionates and elemental sulphur and to a lesser extent in the presence of soluble sulphur compounds, as for example tetrathionate. These same compounds of sulphur are the substrate for the sulfoxidizing activity of these bacteria, where the reduced compounds of sulphur are oxidized to finally produce sulphuric acid. That is to say, the sulfoxidizing activity is correlated with the presence of the spermidine biomarker in the extracellular medium.

The sulfoxidizing activity is of vital importance in many microbiologic processes as for example bioleaching, production of sulphuric acid from elemental sulphur, production of sulfoxidizing biomass, etc. The sulfoxidizing activity is so important in biomining that the incorporation to the process of micro organisms with sulfoxidizing activity is within the usual practice in the bioleaching processes, as Acidithiobacillus thiooxidans, for example by continuous inoculation of the mineral (see patent Biosigma SA. CL 2911-06) or its equivalent (U.S. Pat. No. 7,837,760). Notwithstanding, due to the nature of the mineral, often the sulfoxidizing micro organisms are exposed to toxics that inhibit their activity or eliminate them. For this reason, it is of vital importance to be able to establish rapidly if the sulfoxidizing activity really exists in productive microbiologic processes such as bioleaching, the production of sulphuric acid or the production of sulfoxidizing biomass.

This invention solves this technical problem, since it permits, without the need of quantifying the sulfoxidizing micro organisms to quickly establish if there is sulfoxidizing activity in the process measuring a metabolite secreted in the extracellular medium as in the case of the spermidine biomarker.

Spermidine can be detected by any method available in the state of the art such as chromotography in fine layer (TLC), assay by immune absorption linked to enzymes (ELISA), high performance liquid chromatography (HPLC), colorimetry based on modified fenolftaleine or mass spectrometry, for example. Without prejudice of which, the method preferred by the inventors for its detection is mass spectrometry.

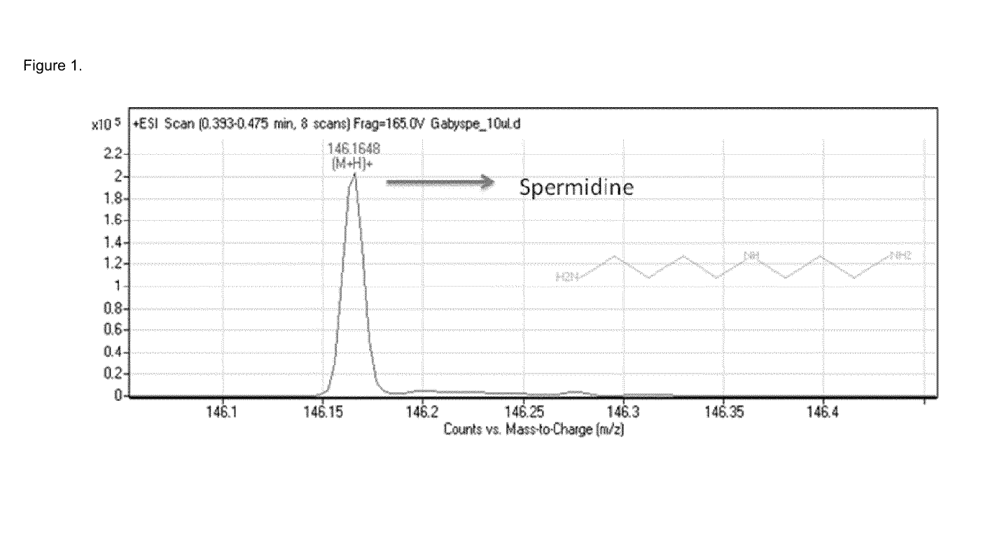

To detect the spermidine in the supernatant or effluent of a microbiologic process by mass spectrometry the sample must be filtered to eliminate the present macromolecules, conveniently with a filter of a size of cut of 3 kDA, subsequently it must be evaporated and suspended in water. The sample thus prepared is detected by means of mass spectrometry, where it is submitted to a gradient of solution A to B, where A is a solution of formic acid 0.1% in water and B is a solution of formic acid 0.1% in acetonitrile. If there is spermidine in the sample, in the spectrum is observed a peak of 146, 1648 m/z, see FIG. 1 where the spectrum of mass spectrometry is shown of a solution of 5 ng/μL of spermidine.

A colorimetric method of detection of spermidine based on modified fenolftaleine that permits the identification at simple sight of spermidine with the characteristic pink color of fenolftaleine is described in the publication of Tanima D, et al (Tanima, D, et al (Tanima D et al, 0 Biomol. Chem. 2009, 7, 4689-4694.)

As we have indicated, the spermidine may be identified by any method available in the present state of the art, or that could be developed in the future, since the method of detection used is not critical in the method of this invention. Where, the intention aims at the meaning of finding an intracellular molecule in the extracellular medium of sulfoxidizing microorganisms, that is, that such molecule spermidine is a biomarker of the sulfoxidizing activity

### EXAMPLES

The presence of spermidine was determined in the effluents of different microbiological processes with or without sulfoxidizing activity.

### Example 1

### Detection of the Spermidine Biomarker in Cultures of Acidithiobacillus thiooxidans and Acidithiobacillus ferrooxidans

1.1) Biomass Production Reactors in Elemental Sulphur with Acidithiobacillus thiooxidans Licanantay DSM 17318.

The means of cultures in different stages of growth of a biomass production reactor were analyzed using elemental sulphur as substrate, for the strain Acidithiobacillus thiooxidans, Licanantay DSM 17318, in this process there is a high sulfoxidizing activity.

The cultures were grown in the KMD 025× media, ((NH₄)₂SO₄, 247.5 mg/L; NaH₂PO₄.H2O, 36.5 mg/L; KH₂PO₄, 13.125 mg/L; MgSO₄.7H2O, 25 mg/L; CaCl₂, 5.25 mg/L), with 1% elemental sulphur, a pH of 1.8, with continuous stirring and at 30° C.

A sample was taken from the culture in exponential growth phase (T1), early stationary phase (T2) and late stationary phase (T3). And analyzed by mass spectrometry by means of the following protocol.

Preparation of the Sample:
A sample of 5 mL is taken from the culture or otherwise from effluent.
The sample is filtered with a filter of the size of a pore of 0.22 μm to collect extracellular medium.

Spermidine Detection by Mass Spectrometry:
10 μL of prepared sample are injected in an HPLC equipment
The sample is run through a reverse phase column Agilent Zorbax Extend C18 (Number of Product 727700-902).
It is subjected to a gradient of A to B solution where A is a solution of formic acid 0.1% in water, and B is a solution of formic acid 0.1% in acetonitrile Time: 0 to 5 minutes.
It is ionized by electro ionization in positive mode and is detected by means of mass spectrometry.
If there is spermidine in the sample, a peak of 146, 1648 m/z is observed in the spectrum.

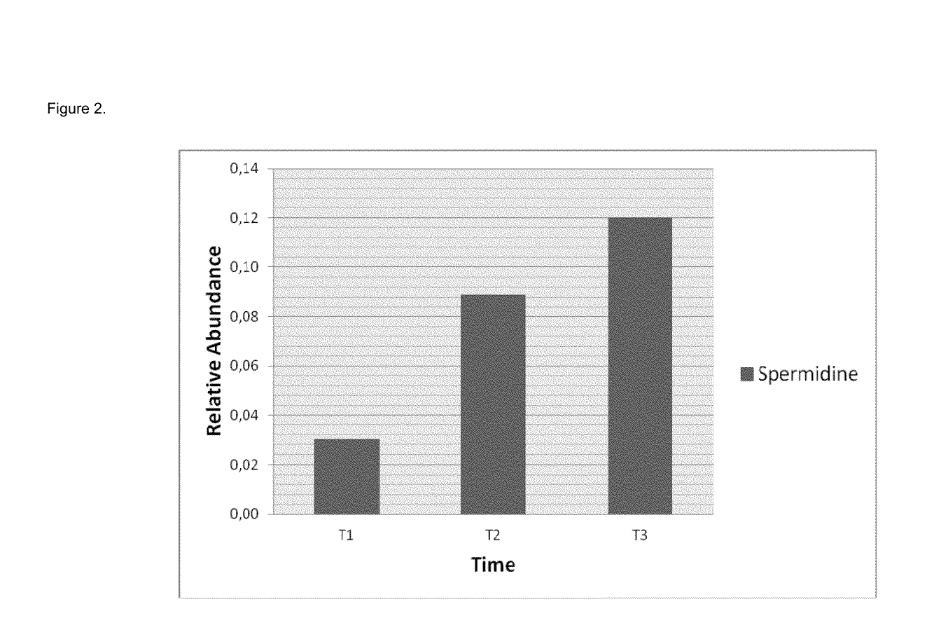

The results show the presence of spermidine in the extracellular medium and can be appreciated in FIG. 2. It is observed that in the 3 stages of the growth of Acidithiobacillus thiooxidans Licanantay DSM 17318 in the presence of elemental sulphur, there is a presence of spermidine in the supernatant, and that the concentration of spermidine is increased in time, being 0.03 units of relative abundance (A.r.) in the exponential phase of the growth (T1) of 0.09 A.r., in the early stationary phase (T2) and of 0.12 A.r. in the late stationary phase (T3).

1.2) Biomass Production Reactors in the Presence of Fe+2 Without Elemental Sulphur with Acidithiobacillus ferrooxidans Wenelen DSM 16786.

The means of cultivation were analyzed in different stages of growth of a biomass production reactor using ferrous sulphate as surface, for the strain Acidithiobacillus ferrooxidans Wenelen DSM 16786, in this process there is no sulfoxidizing activity.

The cultures were grown in the KMD 0.25× media, with 6 g/l, Fe+2 to pH 1.8 with continuous stirring and at 30° C.

A sample was taken from the culture in exponential phase of the growth (T1), early stationary phase (T2) and late stationary phase (T3) and it was analyzed by mass spectrometry using the protocol described in example 1.1, where it was not possible to detect the presence of spermidine in the extracellular medium.

### Example 2

### Detection of the Biomarker Spermidine in the Productive Microbiologic Processes with Sulfoxidizing Activity

### 2.1) Column of Sulphuric Acid Production.

The effluent of a column of sulphuric acid production was analyzed from elemental sulphur. This is a process that is completely sulfoxidizing.

The column of production of sulphuric acid used was 1 meter high, had a diameter of 12 cm. and was loaded with:
12.5 kg of quartz with a size of a range under ⅜ inches (0.4 cm) and higher than ¼ inch (0.63 cm)
1 kg of elemental S (concentration 80 Kg S/ton of quartz)
1*10⁸ cel/g S, of inoculation of Acidithiobacillus thiooxidans Licanantay DSM 17318 and Acidithiobacillus ferrooxidans Wenelen DSM 16786.

The column operates at 20° C. and it is watered with laboratory water at a rate of 1 L/h/m2 in open circuit. During 10 days the irrigation has a pH 3 and on subsequent days the pH was not adjusted, with natural aeration (that is to say, without air injection).

As an average the production of sulphuric acid of the column was 0.6 kg of sulphuric acid/ton of quartz/day. That is, it is verified that the sulfoxidizing activity is present in this column.

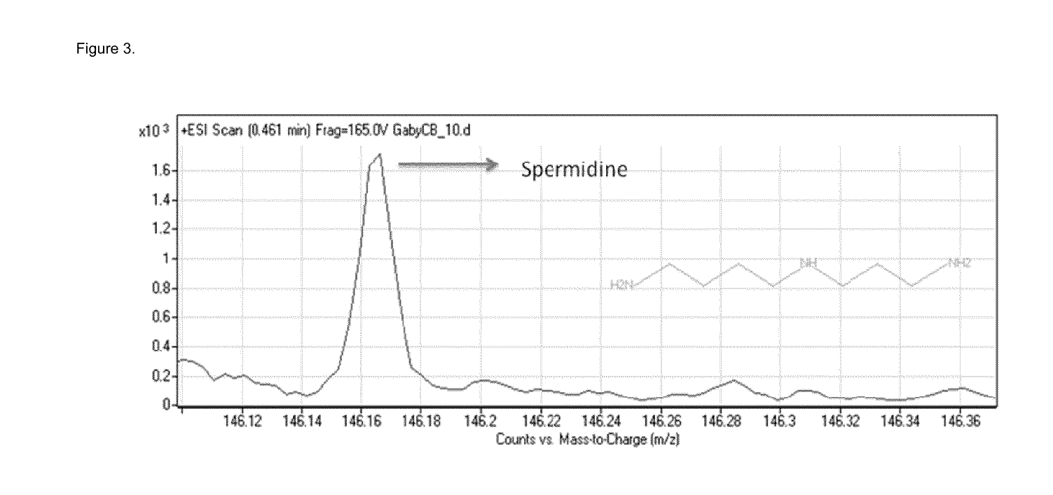

On the 16^{th} day of operation of the column a sample of effluent was taken and analyzed by mass spectrometry, using the protocol described in example 1.1, detecting the presence of spermidine in the effluent. See FIG. 3.

As positive control, 5 ng/μL was added to the effluent of the column and subjected to the same protocol of detection of spermidine described in example 1.1; as was expected the presence of spermidine is observed in the spectrum, see FIG. 1.

### 2.2) Columns of Ferric Ion Production.

The effluent of a ferric ion production column was analyzed, where immobilized bacteria oxidize ferrous ion present in the feeding of the column to ferric ion; in this process there is no important sulfoxidizing activity.

The column of ferric ion production was evaluated, has the following characteristics.

A column of 1 meter high and 12 cm diameter loaded with:

| | |
|---|---|
| Mineral | Leaching sterile material |
| Size | 80% under ½ inch (1.25 cm) |
| Mineral Load | 12 kg |

1*10⁷ cel/g mineral, of inoculation of Acidithiobacillus thiooxidans Licanantay DSM 17318 and Acidithiobacillus ferrooxidans Wenelen DSM 16786.

The column operates at 20° C. and is irrigated with KMD 0.25× media, complemented with 10 g/L Fe+2 at an irrigation rate of 5 L/h/M2 in open circuit. During 38 days the irrigation was at pH 1.4 and on subsequent days the pH was adjusted with natural aeration (that is to say, without the need of air injection.)

On the 39th day a sample of effluent was taken and analyzed by mass spectrometry, using the protocol described in example 1.1, where it was not possible to detect the presence of spermidine in the effluent.

These results show that in the processes in which there is sulfoxidizing activity (examples 1.1 and 2.1) spermidine was detected in the extracellular medium, while in the cases where there is no sulfoxidizing activity (examples 1.2 and 2.2) no spermidine was detected in the extracellular means, In this way the efficacy of the method of the invention was verified that permits evidencing the sulfoxidizing activity of a microbiologic process detecting the presence of the biomarker spermidine produced by the sulfoxidizing micro organisms that participate in such processes.

1. A method to detect the sulfoxidizing activity of a microbiological process, comprising detecting in the extracellular medium the presence of the biomarker spermidine produced by the sulfoxidizing microorganisms that participate in such process.

2. The method in accordance with claim 1 wherein the sulfoxidizing microorganisms present are predominantly of the Acidithiobacillus thiooxidans type.

3. The method in accordance with claim 1 wherein the presence of spermidine is determined in the effluent of the microbiologic process.

4. The method in accordance with claim 3 wherein the microbiological process is a production process of sulphuric acid from elemental sulphur using sulfoxidizing microorganisms.

5. The method in accordance with claim 1 wherein the presence of spermidine is determined in the supernatant of a productive process where sulfoxidizing microorganisms participate.

6. The method in accordance with claim 5 wherein the productive process where sulfoxidizing microorganisms participate is a reactor of biomass production of sulfoxidizing microorganisms.

7. The method in accordance with claim 5 wherein the productive process where sulfoxidizing microorganisms participate is a bioleaching process.

8. The method in accordance with claim 1 wherein the presence of the spermidine biomarker is detected by chromatography in fine layer (TLC), assay for immune absorption linked to enzymes (ELISA), high performance liquid chromatography (HPLC), colorimetry based on modified fenolftaleine or mass spectrometry.
